(19) United States
(12) Patent Application Publication
(10) Pub. No.: US 2025/0266166 A1
(43) Pub. Date: Aug. 21, 2025

(54) CLASSIFICATION DEVICE AND METHOD USING HYPERGRAPH

(71) Applicant: Pusan National University Industry-University Cooperation Foundation, Busan (KR)

(72) Inventors: Giltae Song, Yangsan-si (KR); Ki Beom Kim, Yangsan-si (KR)

(21) Appl. No.: 19/057,967

(22) Filed: Feb. 19, 2025

(30) Foreign Application Priority Data
Feb. 20, 2024 (KR) 10-2024-0024417

(51) Int. Cl. G16H 50/20

(52) U.S. Cl. CPC G16H 50/20

(57) ABSTRACT

Provided are a classification apparatus and a method using a hypergraph for discovery of a therapeutic gene. The apparatus includes a processor and a memory operatively connected to the processor, and the memory stores instructions that, when executed, cause the processor to identify a first hypergraph related to a first target, identify first target embedding based on the first hypergraph, identify a second hypergraph including a part of the first target embedding and related to a second target, identify a second target embedding based on the second hypergraph, identify at least one integrated pair based on the second target embedding, and classify at least one integrated pair based on at least one criterion. The at least one criterion may include unrelated, a biomarker, and a therapeutic gene.

### BACKGROUND OF THIS INVENTION

The present invention relates to a classification device and method using a hypergraph and more particularly, a therapeutic gene identification apparatus and a method thereof using a deep learning capable of classifying a specific target, such as a gene, by a plurality of criteria using a hypergraph.

This section merely provides background information for embodiments of the present invention and do not constitute prior art.

The discovery of biomarker genes plays a significant role in resolving disease pathogenesis and accelerating targeted drug development, along with therapeutic gene intervention and gene therapy. In this regard, various deep learning methods have emerged to accelerate the discovery of gene-disease association (GDA). Recently, these various deep learning methods have focused on learning research on various heterogeneous graph representations, such as genes and diseases, protein interactions, gene ontology, gene expression, and disease ontology.

However, these various deep learning methods have a problem that they depend on prior knowledge and cannot efficiently express complex biological networks, and they are highly dependent on expert knowledge in genetic research and cost time and money.

Specifically, identifying therapeutic genes is crucial for developing treatments targeting genetic causes of diseases, but experimental trials are expensive and time-consuming. Even though many deep learning methods aim to identify biomarker genes, predicting therapeutic target genes remains challenging due to the limited number of known targets.

For example, the use of deep learning for predicting candidate therapeutic gene targets using known disease-gene association information was introduced. This approach has some limitations that it failed to consider the biological ontologies of diseases and genes, which are important for determining the therapeutic potential of genes. Additionally, deep graph representation learning methods which focus on disease-gene associations effectively, while making use of disease-gene and their biological ontologies. Although the deep graph representation learning methods show a capability for predicting genetic markers of diseases, they could not effectively capture one-to-many (meta-path) relationships between biological entities, where a disease is associated with multiple genes, each linked to multiple gene ontologies. This is because an edge in a graph can only represent a one-to-one (heterogeneous) relationship between nodes. In order to effectively define various molecular relationships, meta-path based approaches may be necessary, which may require specialized knowledge in the field of gene research, and a lot of time and money. However, conventional meta-path based approaches rely on limited prior knowledge and have the problem of not being able to efficiently express complex biological networks.

To address this issue, a deep hypergraph learning model which identifies a gene's therapeutic potential, biomarker status, or lack of association with diseases is provided. More specifically, structures of genes, ontologies, diseases, and phenotypes, along with attention-based learning to capture complex relationships are employed.

Compared to the conventional methodologies, the proposed deep hypergraph learning model device has brought improvement on research and development time through faster data processing analysis, which contributed to accelerating gene discovery. Further, the success rate of clinical trials through discovered therapeutic genes has been improved, and more accurate and reliable gene targeting has increased the efficiency and success rate of clinical trials and had a positive impact on patient treatment in the long term. The increase of the accuracy of the classification achieved by this invention can be shown in FIG. 14 of the present application.

Compared to the conventional technology, this proposed invention presents a new methodology that effectively integrates and interprets complex and multidimensional relationships between heterogeneous data such as genes, proteins, and diseases by utilizing hypergraphs. This invention provides a solution that can reduce the dependence on specialized knowledge required in genetic research and reduce the time and cost required to set up complex meta-paths. By using hypergraphs, genetic researchers can analyze genetic interactions in a cheaper and more accessible way. This invention goes beyond simple gene-disease association inference and identifies various characteristics of genes and possible therapeutic targets through multi-class classification.

Edge machine learning (edge ML) is a process of running machine learning algorithms on computing devices at the periphery of a network to make decisions and predictions as close as possible to the originating source of data. It is also referred to as edge artificial intelligence or edge AI. A node may be a fundamental component in artificial intelligence (AI) systems, particularly involving graphs or tree structures. In AI systems where graphs or tree structures are involved, a node may represent a specific data point or element that can be connected to other nodes via edges or links. An edge may include any link or linkage between/among the nodes in the AI systems.

One of the purposes of the present invention is to provide a classification device and a method using a deep hypergraph learning that can classify genes by specific criteria using the deep hypergraph learning. The purposes of the present invention are not limited to the purposes mentioned above, and other purposes and advantages of the present invention that are not mentioned can be understood by the following description, and will be more clearly understood by the embodiments of the present invention. In addition, it will be easily understood that the purposes and advantages of the present invention can be realized by the means and combinations thereof indicated in the patent claims.

A classification device using a deep hypergraph learning according to an embodiment of the present invention includes a processor and a memory operatively connected to the processor, wherein the memory stores instructions that, when executed, cause the processor to identify a first hypergraph related to a first target, identify a first target embedding based on the first hypergraph, identify a second hypergraph including a part of the first target embedding and related to a second target, identify a second target embedding based on the second hypergraph, identify at least one integrated pair based on the second target embedding, and classify the at least one integrated pair based on at least one criterion.

### SUMMARY OF THE INVENTION

A classification device using a deep hypergraph learning according to an embodiment of the present invention includes at least one processor, at least one memory including a computer program code, wherein the computer program code, when executed by the at least one processor, is configured, with the at least one processor, to construct a gene hypergraph associated with genes, the gene hypergraph including first hypernodes and first hyperedges; produce gene embedding vectors by applying a first attention mechanism to the gene hypergraph, wherein the first attention mechanism is configured to update each of the first hypernodes and the first hyperedges; construct a disease hypergraph including one or more of the gene embedding vectors, second hypernodes and second hyperedges, and associated with diseases; produce disease embedding vectors which include information associated with the genes and the diseases by applying a second attention mechanism to the disease hypergraph, wherein the second attention mechanism is configured to update each of the second hypernodes and the second hyperedges; and identify at least one gene-disease pair based on the disease embedding vectors, wherein the computer program code is configured to cause the at least one processor to classify the at least one gene-disease pair based on a criterion including unrelated, a biomarker, and a therapeutic gene.

Further, the computer program code can be configured to cause the at least one processor to construct the gene hypergraph based on a plurality of graph sources including nodes representing gene information and gene ontology information, wherein the nodes are connected by an edge in the plurality of graph sources.

Further, the computer program code can be configured to cause the processor to construct the disease hypergraph based on the plurality of graph sources; produce one or more of the gene embedding vectors associated with at least one of the second hyperedges, wherein the at least one of the second hyperedges is related to the genes represented in the disease hypergraph; and construct the disease hypergraph based on the disease hypergraph and the one or more of the gene embedding vectors.

Further, the computer program code can be configured to cause the processor to update the first hyperedges based on first node information associated with a common relationship of the first hypernodes included in the respective first hyperedges, and produce pre-gene embedding vectors by updating the first hypernodes included in the gene hypergraph based on updated first hyperedge information.

Further, the computer program code can be configured to cause the processor to produce integrated embedding vectors for a gene among the genes, wherein information of the gene is included in both one of the first hypernodes and one of the first hyperedges; and produce the gene embedding vectors based on the integrated embedding vectors and the pre-gene embedding vectors.

Further, the gene embedding vectors can further include information associated with the gene hypergraph.

Further, the computer program code can be configured to cause the processor to update the second hyperedges based on second node information associated with a common relationship of the second hypernodes included in the respective second hyperedges of the disease hypergraph; and produce the disease embedding vectors by updating the second hypernodes based on updated second hyperedge information.

Further, a classification apparatus using a hypergraph according to another embodiment of the present invention can include at least one processor; and at least one memory including a computer program code, wherein the computer code, when executed by the at least one processor, is configured, with the at least one processor, to construct a gene hypergraph associated with genes, the gene hypergraph including first hypernodes and first hyperedges; update the first hyperedges included in the gene hypergraph based on first node information associated with a common relationship of the first hypernodes included in the respective first hyperedges; produce pre-gene embedding vectors by updating the first hypernodes included in the gene hypergraph based on updated first hyperedge information; produce integrated embedding vectors for a gene among the genes, wherein information of the gene is included in both one of the first hypernodes and one of the first hyperedges; produce gene embedding vectors based on the integrated embedding vectors and the pre-gene embedding vectors; construct a disease hypergraph including one or more of the gene embedding vectors, second hypernodes, and second hyperedges, and associated with diseases; update the second hyperedges based on second node information associated with a common relationship of the second hypernodes included in the respective second hyperedges of the disease hypergraph; produce disease embedding vectors including information associated with the genes and the diseases by updating the second hypernodes based on updated second hyperedge information; identify at least one gene-disease pair based on the disease embedding; and classify the at least one gene-disease pair based on at least one criterion including unrelated, a biomarker, and a therapeutic gene.

Further, the computer program code can be configured to cause the processor to construct the gene hypergraph based on a plurality of graph sources including nodes representing gene information and gene ontology information, wherein the nodes are connected by an edge in the plurality of the graph sources.

Further, the computer program code can be configured to cause the processor to construct the disease hypergraph based on the plurality of graph sources; produce one or more of the gene embedding vectors associated with at least one of the second hyperedges, wherein the at least one of the second hyperedges is related to the genes represented in the disease hypergraph; and construct the disease hypergraph based on the disease hypergraph and the one or more of the gene embedding vectors.

Further, the gene embedding vectors can further include information related to the gene hypergraph.

Further, a therapeutic gene identification method using a hypergraph according to an embodiment of the present invention can include identifying a gene hypergraph associated with genes, the gene hypergraph including first hypernodes and first hyperedges; identifying gene embedding vectors by applying a first attention mechanism to the gene hypergraph, wherein the first attention mechanism is configured to update each of the first hypernodes and the first hyperedges; identifying a disease hypergraph including one or more of the gene embedding vectors, second hypernodes, and second hyperedges, and associated with diseases; identifying disease embedding vectors including information associated with the genes and the diseases by applying a second attention mechanism to the disease hypergraph, wherein the second attention mechanism is configured to update each of the second hypernodes and the second hyperedges; identifying at least one gene-disease pair based on the disease embedding vectors; and classifying the at least one gene-disease pair based on a criterion including unrelated, a biomarker, and a therapeutic gene.

Further, the step of identifying the gene hypergraph can be performed based on a plurality of graph sources including nodes representing gene information and gene ontology information, wherein the nodes are connected by an edge in the plurality of graph sources.

Further, the step of identifying the disease hypergraph can be performed based on the plurality of graph sources, and the identifying of the disease hypergraph includes: identifying one or more of the gene embedding vectors associated with at least one of the second hyperedges, wherein the at least one of the second hyperedges is related to the genes represented in the disease hypergraph; and identifying the disease hypergraph based on the disease hypergraph and the one or more of the gene embedding vectors.

Further, the step of identifying of the gene embedding vectors can further include applying the first attention mechanism so as to: update the first hyperedges based on first node information associated with a common relationship of the first hypernodes included in the respective first hyperedges; and identify pre-gene embedding vectors by updating the first hypernodes included in the gene hypergraph based on updated first edge information.

Further, the step of identifying of the gene embedding vectors can include: identifying integrated embedding vectors for a gene among the genes, wherein information of the gene is included in both one of the first hypernodes and one of the first hyperedges; and identifying the gene embedding vectors based on the integrated embedding vectors and the pre-gene embedding vectors.

Further, the therapeutic gene identification method can further include a step of including information associated with the gene hypergraph to the gene embedding vectors.

Further, the step of identifying the disease embedding vectors can include the step of applying the second attention mechanism so as to: update the second hyperedges based on second information associated with a common relationship of the second hypernodes included in the respective second hyperedges of the disease hypergraph; and identify the disease embedding vectors by updating the second hypernodes based on updated second edge information.

The therapeutic gene identification apparatus (classification apparatus) and the method using a hypergraph according to the present invention can reduce the dependence on specialized knowledge required for genetic research and reduce time and cost by classifying genes according to specific criteria using a hypergraph.

Further the classification apparatus and method using the hypergraph of the present invention classifies genes by specific criteria using the hypergraph, thereby going beyond simple inference of a correlation between genes and diseases, and discovering various characteristics of genes through detailed classification of genes and identifying possible treatment targets.

Further, the classification apparatus and method using the hypergraph of the present invention can improve the accuracy of discovery of therapeutic genes and improve the accuracy of identification of potential therapeutic targets by classifying genes based on specific criteria using the hypergraph.

Further, the classification apparatus and method using the hypergraph of the present invention can shorten research and development time by improving the efficiency of data processing and analysis related to gene classification by classifying genes according to specific criteria using the hypergraph, and can contribute to the acceleration of discovery of therapeutic genes.

Further, the classification apparatus and method using the hypergraph of the present invention can improve the possibility of success of clinical trials using classified therapeutic genes by classifying genes based on specific criteria using the hypergraph, improve the efficiency of clinical trials through more accurate and reliable gene targeting, and have a positive effect on patient treatment.

Further, the classification apparatus and method using the hypergraph of the present invention classifies genes by specific a criteria using the hypergraph, thereby making the meta path necessary for defining relationships between various molecules (e.g., nodes such as genes and diseases) unnecessary, and thus complex and multidimensional relationships between heterogeneous data such as genes and diseases can be effectively integrated and interpreted.

In addition to the above-described contents, the specific effects of the present invention are described together with the specific matters for carrying out the invention below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram for explaining a classification apparatus using a hypergraph according to an embodiment of the present invention.

FIGS. 2 and 3 are diagrams for explaining a processor of a classification apparatus using a hypergraph according to an embodiment of the present invention.

FIG. 4 is a flowchart for explaining the operation of a processor of a classification apparatus using a hypergraph according to an embodiment of the present invention.

FIGS. 5A-5B are diagrams for explaining step S100 of FIG. 4.

FIGS. 6 and 7 are flowcharts for explaining step S200 of FIG. 4.

FIGS. 8 and 9 are diagrams for explaining step S300 of FIG. 4.

FIGS. 10 and 11 are flowcharts for explaining step S400 of FIG. 4.

FIG. 12 is a flowchart for explaining a classification method using a hypergraph according to an embodiment of the present invention.

FIGS. 13 to 15 show a table and diagrams for explaining the effects of a classification apparatus and a method using a hypergraph according to an embodiment of the present invention.

### DETAILED DESCRIPTION OF THE INVENTION

In the following detailed description, numerous specific details are set forth in order to provide a thorough understanding of embodiments. However, it will be understood by those of ordinary skill in the art that the embodiments may be practiced without these specific details. In other instances, well-known methods, procedures, components and circuits have not been described in detail so as not to obscure the embodiments.

The following description with reference to the accompanying drawing illustrates specific embodiments to enable those skilled in the art to practice them. Other embodiments may incorporate structural, logical, process, and other changes. Portions and features of some embodiments may be included in, or substituted for, those of other embodiments. Embodiments set forth in the claims encompass all available equivalents of those claims. The example embodiments are presented for illustrative purposes only and are not intended to be restrictive or limiting on the scope of the disclosure or the claims presented herein.

The functions described herein may be implemented in software in one embodiment. The software may consist of computer executable instructions stored on computer readable media or computer readable storage devices such as one or more non-transitory memories or other type of hardware-based storage devices, either local or networked.

Although the following description uses terms “first,” “second,” and the like and “A”, “B”, and the like to describe various elements, these elements should not be limited by the terms. The terms are used only to distinguish one element from another. For example, without departing from the scope of the present invention, the first element may be referred to as the second element, and similarly, the second element may also be referred to as the first element.

The terminology used in the description of the embodiments herein is for the purpose of describing a particular embodiment only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof. Throughout the specification, when an element is referred to as being “connected or coupled” to another element, it can be directly connected or coupled to the other element or intervening elements may be present.

The terms or words used in this specification and the claims should not be interpreted as limited to their general or dictionary meanings. In accordance with the principle that the inventor can define the concept of a term or word in order to best explain his or her invention, they should be interpreted as meanings and concepts that are consistent with the technical idea of the present invention. In addition, the embodiments described in this specification and the configurations illustrated in the drawings are only one embodiment in which the present invention is realized, and do not represent the entire technical idea of the present invention, so it should be understood that there may be various equivalents, modifications, and applicable examples that can replace them at the time of this application.

The terminology used in this specification and claims is for the purpose of describing specific embodiments only and is not intended to limit the present invention. The term “and” “or” or “and/or” includes any combination of a plurality of related listed items or any item among a plurality of related listed items. A singular expression includes a plural expression unless the context clearly indicates otherwise. The plural expressions may include a singular expression unless otherwise indicated. It should be understood that the terms “comprise” “include” or “have” in this application do not exclude in advance the possibility of the presence or addition of features, numbers, steps, operations, components, parts or combinations thereof described in the specification.

Unless otherwise defined, all terms used herein, including technical or scientific terms, have the same meaning as commonly understood by one of ordinary skill in the art to which this invention belongs.

Terms defined in commonly used dictionaries should be interpreted as having a meaning consistent with the meaning they have in the context of the relevant technology, and shall not be interpreted in an ideal or overly formal sense unless explicitly defined in this application. In addition, each configuration, process, process, or method included in each embodiment of the present invention may be shared within a scope that is not technically contradictory to one another.

Identifying therapeutic genes is crucial for developing treatments targeting genetic causes of diseases, but experimental trials are expensive and time-consuming. Even though many deep learning approaches aim to identify biomarker genes, predicting therapeutic target genes remains challenging due to the limited number of known targets. To address this issue, a deep hypergraph learning model which identifies a gene's therapeutic potential, biomarker status, or lack of association with diseases is provided. More specifically, structures of genes, ontologies, diseases, and phenotypes, along with attention-based learning to capture complex relationships are employed.

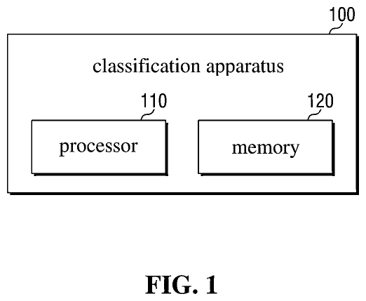

Hereinafter, a classification apparatus and method using a hypergraph according to an embodiment of the present invention will be described with reference to FIGS. 1 to 15.

FIG. 1 is a diagram for explaining a classification apparatus using a hypergraph according to an embodiment of the present invention.

Referring to FIG. 1, a classification apparatus (100) using a hypergraph according to an embodiment of the present invention can include a processor (110) and a memory (120).

The classification apparatus (100) can be supplemented with one or more other components, such as a communication module. In some embodiments, some of these components can be implemented as a single integrated circuit.

The memory (120) can store various data used by at least one component (e.g., processor (110)) of the classification apparatus (100). The data can include, for example, input data or output data for software (e.g., program) and commands related thereto. The memory (120) can include volatile memory or nonvolatile memory.

The memory (120) can store commands, information, or data associated with the operation of components included in the classification apparatus (100). For example, the memory (120) can store instructions that, when executed, enable the processor (110) to perform various operations described in this document.

The processor (110) can be operatively coupled with a memory (120) to perform the overall function of the classification apparatus (100). The processor (110) can include, for example, one or more processors. The one or more processors can include, for example, an image signal processor (ISP), an application processor (AP), or a communication processor (CP).

The processor (110) can, for example, execute software (e.g., a program) to control at least one other component (e.g., a hardware or software component) of the classification apparatus (100) connected to the processor (110), and can perform various data processing or calculations. According to one embodiment, as at least a part of the data processing or calculations, the processor (110) can load a command or data received from another component (e.g., a communication module) into the memory (120), process the command or data stored in the memory (120), and store result data in the memory (120). According to another embodiment, the processor (110) can include a main processor (e.g., a central processing unit or an application processor), and an auxiliary processor (e.g., a graphics processing unit (GPU), an image signal processor, a sensor hub processor, or a communication processor) that can operate independently or together therewith. Additionally, or alternatively, the auxiliary processor can be configured to use less power than the main processor, or to be specialized for a given function. The auxiliary processor can be implemented separately from the main processor, or as a part thereof. The program can be stored as software in memory (120) and can include, for example, an operating system, middleware, or an application.

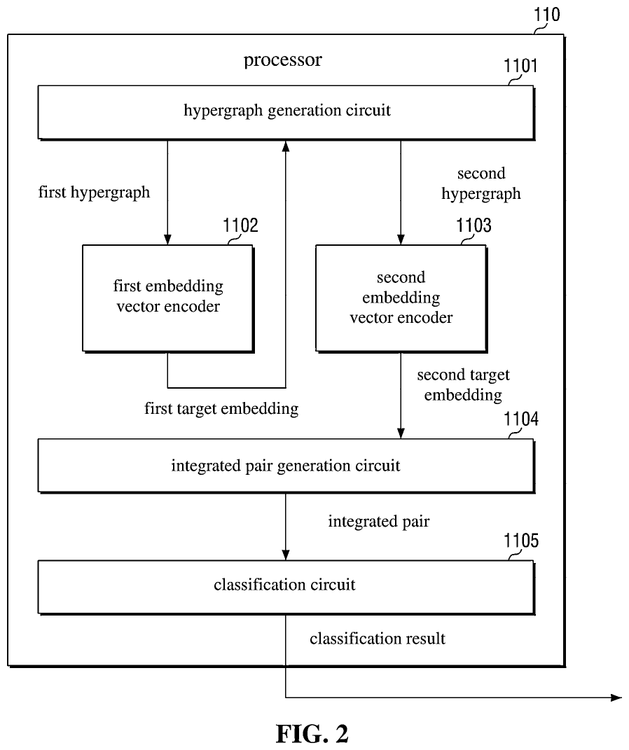

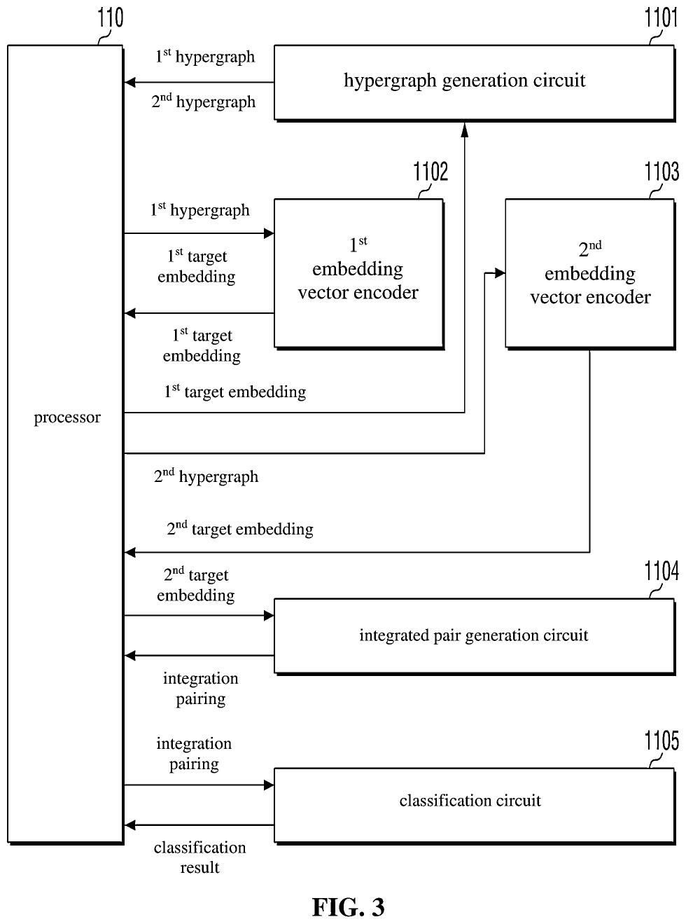

FIG. 2-3 are diagrams for explaining a processor of a classification apparatus using a hypergraph according to an embodiment of the present invention.

Referring to FIGS. 2 and 3, a processor (110) of a classification apparatus (100) using a hypergraph according to an embodiment of the present invention can include a hypergraph generation circuit (1101), a first embedding (vector) encoder (1102), a second embedding (vector) encoder (1103), integrated pair generation circuit (1104), and a classification circuit (1105).

In some embodiments, as shown in FIG. 3, the hypergraph generation circuit (1101), the first embedding (vector) encoder (1102), the second embedding (vector) encoder (1103), the integrated pair generation circuit (1104), and the classification circuit (1105) can be located outside the processor (110).

Embedding can be defined as a low-dimensional, learned continuous vector representation of discrete variables into which a user can translate high-dimensional vectors. Embedding can be also defined as a method to represent a categorical variable using some real numbers or mathematically defined as a vector. Accordingly, gene embedding can be defined as numerical representations of each of genes using numbers and/or vectors that machine learning and artificial intelligence systems use for understanding and learning, or a method thereof. In the same way, disease embedding can be defined as numerical representations of each of diseases using numbers and/or vectors, or a method thereof.

The operation of the processor (110) in FIG. 2 is the same as the operation of the processor (110) in FIG. 3. The operation of the processor (110) is described below with reference to FIGS. 1 to 4.

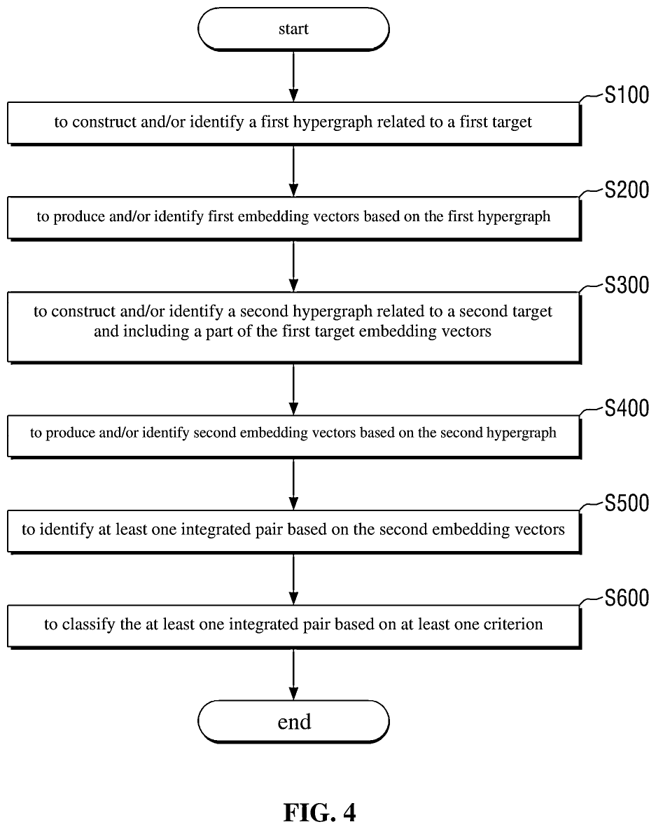

FIG. 4 is a diagram for explaining the operation of a processor of a classification apparatus using a hypergraph according to an embodiment of the present invention.

Referring to FIGS. 1-4, the processor (110) (e.g., hypergraph generation circuit (1101)) can construct and/or identify a first hypergraph related to a first target (S100). For example, the processor (110) (e.g., hypergraph generation circuit (1101)) can construct and/or identify a gene hypergraph related to a gene. It is noted that, in the present application, the terms to “construct” and/or “produce” a hypergraph and/or embedding may be necessary for a process of “identifying” the hypergraph and/or embedding. It is also noted that the first target may correspond to gene(s) and the second target may correspond to disease(s).

The processor (110) (e.g., hypergraph generation circuit (1101)) can generate a first hypergraph based on a plurality of graph sources. The plurality of graph sources can include a graph in which first information is configured as and/or includes a first node, second information is configured as and/or includes a second node, and the first node and the second node are connected by an edge.

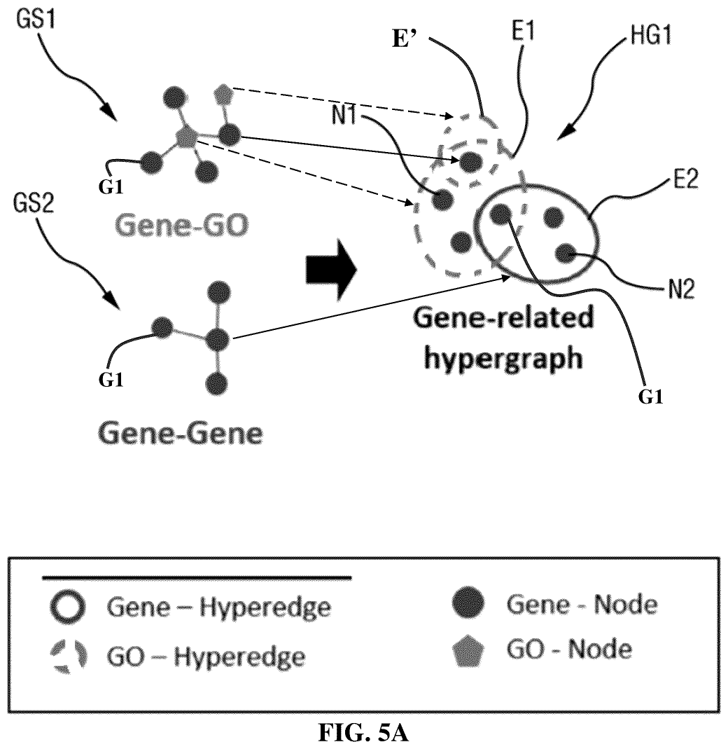

FIGS. 5A-5B are diagrams for explaining step (S100) of FIG. 4. FIG. 5A illustrates an example of a first hypergraph.

Referring to FIGS. 1 to 5A-5B, a plurality of graph sources can include, for example, a first graph source (GS1) and a second graph source (GS2). The plurality of graph sources can be related to, for example, a first target. The plurality of graph sources can be associated with, for example, a gene.

As shown in FIG. 5A, a first graph source (GS1) can include a gene as first information and gene ontology (GO) as second information, respectively, as nodes, and the first information and the second information are connected with an edge. In other words, it can be illustrated that a gene node is connected to a GO node by a single edge as shown in GS1. Also, as shown in FIG. 5A, a second graph source (GS2) can include the first information—gene(s) as node(s), and one of the first information (gene(s)) and another one of the first information (gene(s)) are connected with an edge. In other words, it can be illustrated that a gene node is connected to another gene node by a single edge as shown in GS2.

A processor (110) (e.g., a hypergraph generation circuit (1101)) can construct and/or identify a first hypergraph (e.g., a gene-related hypergraph) (HG1) associated with a first target (e.g., a gene—particularly, a target gene) based on a plurality of graph sources (GS1, GS2) related to the first target.

As illustrated in FIG. 5A, a first hypergraph (HG1) can include node(s) and hyperedge(s). In the present application, for clarification, a node and an edge included in a hypergraph may be respectively referred to as a hypernode and a hyperedge. The hypernodes of the first hypergraph (HG1) can include a first hypernode (N1) and a second hypernode (N2). The hypernodes of the first hypergraph (HG1) can be related to the first target (e.g., a gene). The hyperedges of the first hypergraph (HG1) can include at least one type of a target. The hyperedges of the first hypergraph (HG1) can include a hyperedge E1 and a hyperedge E2. The hyperedges of the first hypergraph (HG1) can be associated with a first target (e.g., a gene). For example, the hyperedges of the first hypergraph (HG1) can include genes and GO.

A hyperedge includes at least one node. Different hyperedges can include a common node. For example, a hyperedge E1 can include four nodes including a node N1. For example, a hyperedge E2 can include three nodes including a node N2. For example, the hyperedge E1 and the hyperedge E2 in FIG. 5A can have one node in common.

For example, as illustrated in FIG. 5A, the gene ontology (GO) nodes in the first graph source (GS1) can be converted into the GO hyperedges (E1 and E′) as shown in FIG. 5A. The GO node having four edges in GS1 can be converted into the hyperedge encompassing four gene hypernodes (E1), which indicates that the four nodes connected to the corresponding GO-node are included by the hyperedge E1. It is shown by a lower broken-lined arrow in FIG. 5A. Similarly, the GO node having one edge in GS1 can be converted into the hyperedge (E3) encompassing one gene hypernode such that E3 may indicate the one node connected to the corresponding GO-node is included by the hyperedge E3. It is also shown by an upper broken-lined arrow in FIG. 5A. The gene-node connected by both GO-nodes in GS1 can be drawn to be a hypernode included by both GO-hyperedges (E1 and E′) in HG1 (see the upper solid-lined arrow in FIG. 5A). Similarly, the gene node connected to the other gene nodes with three edges in GS2 can be converted into the gene hyperedge (E2), which is shown by a lower solid-lined arrow in FIG. 5A. Further, both of the graph source 1 and the graph source 2 may contain a gene node G1, and G1 may be included by both E1 and E2. The hypernodes included in the same hyperedge may be understood as having the common relationship as they are categorized by the same hyperedge.

FIG. 5B illustrates another example, in which gene node G1′, G2, and G3 are both included by the first graph source (GS_{A}) and the second graph source (GS_{B}). Also, G1′ is connected to G2, G3 and G5 according to GS_{B}. In such a case, G_{A} may be expressed as a hyperedge E_{C} (converted from G1′ of GS_{A}) as well as a hypernode G1′ (converted from G1′ of GS_{A}) in the hypergraph (HG_{A}). In other words, in this example, G1′ in GS_{A} and GS_{B} may correspond to both of a hypernode and a hyperedge in HG_{A}. In this example, as G1′ may be seen as a hypernode and as a hyperedge at the same time in HG_{A}, and thus, it may be necessary to integrate a representation of G1′ from a hypernode perspective and a representation of G1′ from a hyperedge perspective. This integration of embedding process can achieve the accurate representation of the G1′ information.

Referring again to FIGS. 1-4, the processor (110) can construct and/or identify a first hypergraph and input the first hypergraph to a first embedding encoder (1102). The processor (110) (e.g., the first embedding encoder (1102)) can produce and/or identify first target embedding based on the first hypergraph (S200). For example, the processor (110) (e.g., the first embedding encoder (1102)) can produce and/or identify gene embedding based on a gene hypergraph.

A processor (110) (e.g., a first embedding encoder (1102)) can produce and/or identify first target embedding by applying a first attention mechanism that updates each hypernode and hyperedge included in the first hypergraph to the first hypergraph.

An attention mechanism can be defined as a machine learning technique which directs a deep learning AI system to prioritize the most relevant parts of input data. In other words, the attention mechanism does not treat all words in an input sentence with the same weight, but gives more weight to input words corresponding to specific positions in an output sentence, which allows the model to operate more accurately and flexibly even when the lengths of the input and output are different.

An intra-mutual mechanism may be referred to as a process comparing different positions in an input sequence to generate representations. The intra-mutual attention mechanism can aggregate the information of hypernodes connected by hyperedges based on the relative attention weights of the hypernodes to each target hyperedge, and define the hyperedge embedding vectors by combining the information of the hypernodes.

An inter-mutual attention mechanism can be referred to as a computational mechanism that aggregates hyperedge in a gene or disease hypergraph and updates the information of one or more hypernodes. The inter-mutual attention mechanism can receive relationship information between multiple hypernodes from the hyperedge and reflects it to each hypernode.

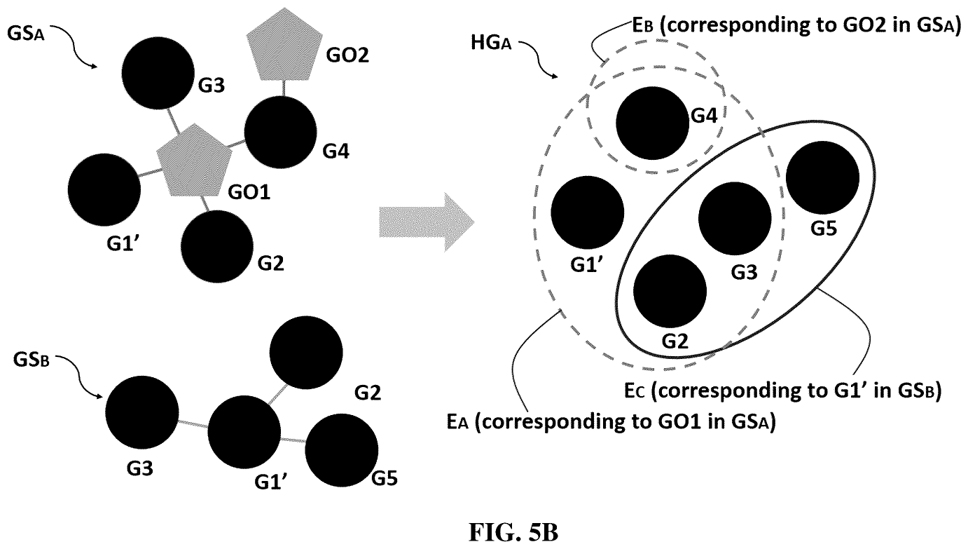

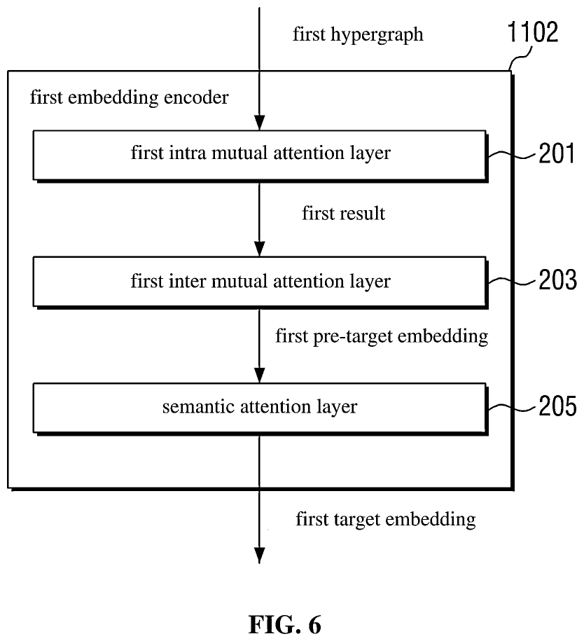

FIG. 6-7 are diagrams for explaining step S200 of FIG. 4.

Referring to FIGS. 1-7, a processor (110) (e.g., a first embedding encoder (1102)) can input a first hypergraph (HG1) to a first intra mutual attention layer (201) to identify a first result.

The processor (110) (e.g., the first intra mutual attention layer (201) of the first embedding encoder (1102)) can identify a common relationship of at least one hypernode included in a hyperedge of the first hypergraph (HG1) and update the hyperedge based on hypernode information related to the common relationship (S201). In other words, the processor (110) (e.g., the first intra mutual attention layer (201) of the first embedding encoder (1102)) can integrate information of a node included in a hyperedge and update information of the hyperedge based on information of the integrated node. For clarification, the common relationship based on the hypernode information can found between certain hypernodes if the hypernodes are included in the same hyperedge of the hypergraph. For example, in FIG. 5B, a common relationship among G1′, G2, G3 and G4 embedding vectors can be found based on the hyperedge E_{A}, a common relationship among G4 embedding vectors can be found based on the hyperedge E_{B}, and a common relationship among G2, G3 and G5 embedding vectors can be found based on the hyperedge E_{C}.

For example, the processor (110) (e.g., the first intra mutual attention layer (201) of the first embedding encoder (1102)) can identify first node information related to a common relationship between four nodes included in the hyperedge E1 and update the hyperedge E1 based on the first node information. The four nodes can be different genes. The processor (110) (e.g., the first intra mutual attention layer (201) of the first embedding encoder (1102)) can update each of at least one hyperedge included in the first hypergraph (HG1) based on the first node information to output the first result.

Layer normalization may be understood as a technique used in machine learning and AI to normalize the inputs of a neural network layer. A normalization layer mechanism may be a computational mechanism, for individual data embedding, that performs numerical normalization among embedding vector elements. The normalization layer mechanism can further stabilize the machine learning.

A linear layer may be understood as a computational algorithm that applies a linear layer mechanism (linear transformation) to the computational results of the attention mechanism to match the input vector dimensions and output vector dimensions of the attention mechanism.

In some embodiments, the first embedding encoder (1102) can include a first normalization layer and a first linear layer. For example, a first hypergraph (HG1) input to the first embedding encoder (1102) can be normalized through the first normalization layer, input to the first intra mutual attention layer (201), and output as a first result through the first linear layer.

The processor (110) (e.g., the first embedding encoder (1102)) can input the first result to a first inter mutual attention layer (203) to produce and/or identify the first pre-target embedding.

The processor (110) (e.g., the first inter mutual attention layer (203) of the first embedding encoder (1102)) can update a node of the first hypergraph (HG1) included in the first result. The processor (110) (e.g., the first inter mutual attention layer (203) of the first embedding encoder (1102)) can produce and/or identify the first pre-target embedding by updating at least one node included in the first hypergraph (HG1) based on the first node information (S203).

In some embodiments, the first embedding encoder (1102) can include a second normalization layer and a second linear layer. For example, the first result can be normalized through the second normalization layer, input to the first inter mutual attention layer (203), and output as a first pre-target embedding through the second linear layer.

The processor (110) (e.g., the first embedding encoder (1102)) can produce and/or identify the first target embedding by inputting the first pre-target embedding into a semantic attention layer (205).

The processor (110) (e.g., the semantic attention layer (205) of the first embedding encoder (1102)) can produce and/or identify integrated embedding for the first target type based on that the first target type corresponds to both one of the hypernode and one of the hyperedge among the first targets included in the first pre-target embedding (S205). For example, as illustrated in FIG. 5B, gene information may be represented in two forms in a hypergraph, for example, hypernode(s) and hyperedge(s), at the same time. The attention mechanism that integrates the representations based on their relative importance is the ‘semantic attention mechanism’. Therefore, the processor (110) (e.g., the semantic attention layer (205) of the first embedding encoder (1102)) can produce and/or identify integrated embedding for a particular gene among the genes using information of the gene included in both the one of the hypernodes and one of the hyperedges included in the first hypergraph (HG1). For example, there may be a case where gene information A is used as both a node and a hyperedge in the process of implementing a hypergraph. That is, gene information A may be used as a node in the gene hypergraph and, at the same time, as a hyperedge connecting gene information B, C and D. For instance, if there is gene information A, B, C, D, E, and the like, gene information A and D may serve as both a node and a hyperedge, and gene information B, C, and the like is only used as nodes. In such a case, gene information A and D may be referred to as ‘a gene included in a hypergraph corresponding to both a node and a hyperedge’. The integrated embedding can be produced by applying a semantic attention mechanism that integrates the semantic information contained in the two representations based on their importance.

A semantic attention mechanism may refer to an operational mechanism that weights and integrates the semantic information contained in the two representations based on their importance. For example, if the node representation is 0.6 and the hyperedge representation is 0.4 when representing a certain gene A, the node representation is relatively more important than the hyperedge representation, so it is calculated as [0.6×node information (vector)]+[0.4×hyperedge information (vector)]=[single representation (vector) of gene A].

The processor (110) (e.g., the semantic attention layer (205) of the first embedding encoder (1102)) can produce and/or identify first target embedding based on the integrated embedding and the first pre-target embedding (S207). The first target embedding can be, for example, gene embedding.

In some embodiments, the processor (110) can further include information related to the first hypergraph in the first target embedding. For example, the first target embedding can be information processed from the first hypergraph, which is the initial information. Therefore, by adding information related to the first hypergraph, which is the initial information, to the first target embedding, the accuracy of information between nodes and hyperedges can be improved.

Referring again to FIGS. 1-4, the processor (110) (e.g., the hypergraph generation circuit (1101)) can construct and/or identify a second hypergraph that includes a part of the first target embedding and is related to a second target (S300). For example, the processor (110) (e.g., the hypergraph generation circuit (1101)) can construct and/or identify a disease hypergraph that includes a part of the gene embedding (one or more of gene embedding among the entire gene embedding vectors) and is associated with a disease.

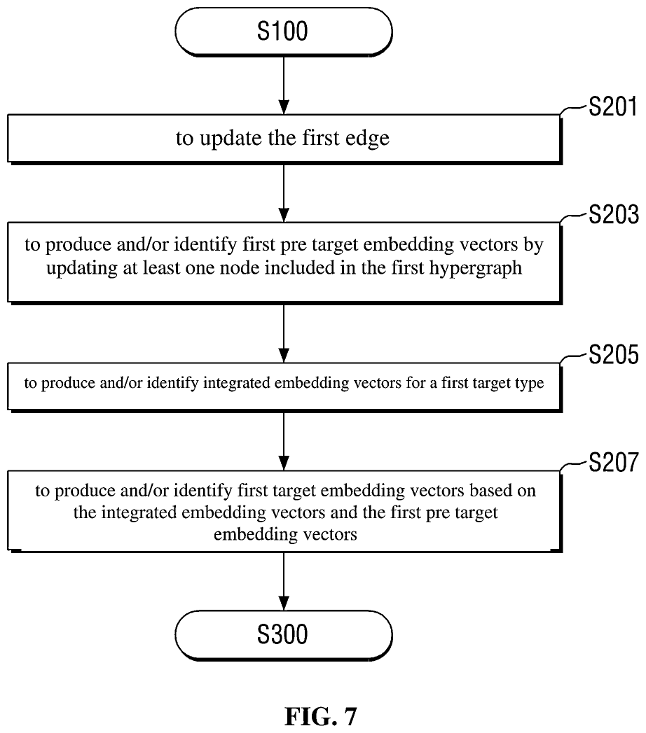

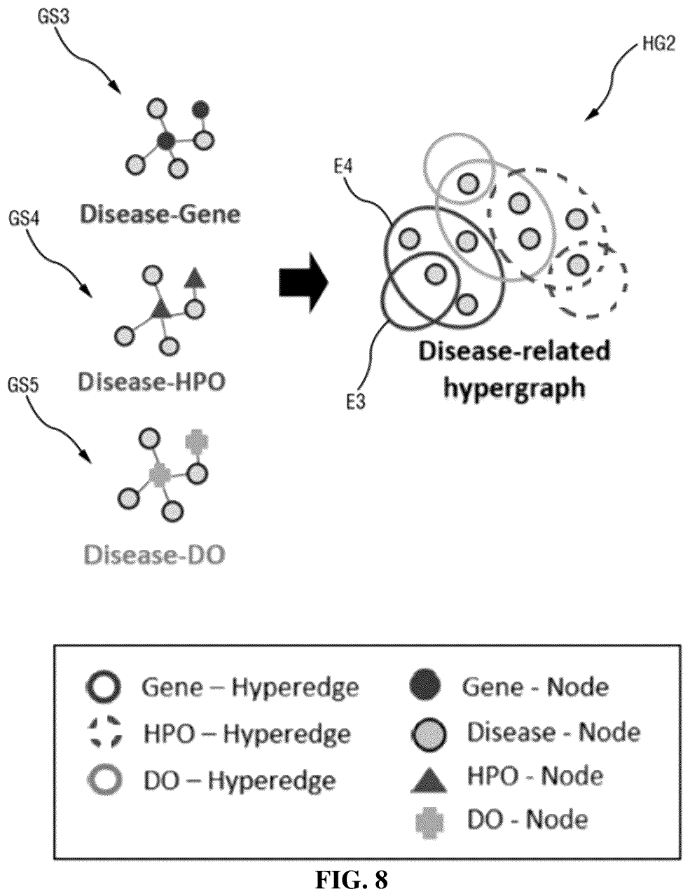

FIGS. 8-9 are drawings for explaining step S300 of FIG. 4.

Referring to FIGS. 1-5, 8, and 9, a processor (110) (e.g., a hypergraph generation circuit (1101)) can construct and/or identify a second hypergraph based on a plurality of graph sources (S301). The plurality of graph sources can include, for example, a third graph source (GS3), a fourth graph source (GS4), and a fifth graph source (GS5). The plurality of graph sources can be related to, for example, a second target. The plurality of graph sources can be related to, for example, a disease.

Specifically referring to FIG. 8, the third graph source (GS3) is configured such that the third information which can be disease(s) and the first information which can be gene(s) are each configured as nodes, and the first information (e.g., gene(s)) and the third information (e.g., disease(s)) can be connected by an edge. The fourth graph source (GS4) is configured such that the third information and the fourth information which can be human phenotype ontology (HPO) are each configured as nodes, and the third information and the fourth information (e.g., HPO) can be connected by an edge. The fifth graph source (GS5) is configured such that the third information and the fifth information which can be disease ontology (DO) are each configured as nodes, and the third information and the fifth information (e.g., DO) can be connected by an edge.

The processor (110) (e.g., hypergraph generation circuit (1101)) can construct and/or identify a second hypergraph (e.g., disease hypergraph) related to the second target based on a plurality of graph sources (GS3, GS4, GS5) related to the second target (e.g., disease(s)).

The processor (110) (e.g., the hypergraph generation circuit (1101)) can construct and/or identify a hyperedge (more specifically, a hyperedge included in the second hypergraph) related to the first target included in the second hypergraph, and produce and/or identify a part of the first target embedding (one or more of the first target embedding vectors among the entire first target embedding vectors) related to the identified hyperedge (S303). For example, the processor (110) (e.g., the hypergraph generation circuit (1101)) can produce and/or identify hyperedge(s) (E3, E4) related to genes represented in the second hypergraph, and produce and/or identify a part (one or more) of the first target embedding associated with the hyperedge(s) (E3, E4).

The processor (110) (e.g., the hypergraph generation circuit (1101)) can identify a second hypergraph (HG2) based on the second hypergraph and a part of the first target embedding (one or more of the first target embedding vectors among the entire first target embedding vectors) (S305). For example, the processor (110) (e.g., the hypergraph generation circuit (1101)) can identify a second hypergraph (e.g., a disease-related hypergraph) (HG2) based on the second hypergraph and a part of the first target embedding (one or more of the first target embedding vectors among the entire first target embedding vectors) already generated. For example, the second hypergraph (HG2) can include a part of the first target embedding (one or more gene embedding vectors among the entire gene embedding vectors).

Referring again to FIGS. 1-4, the processor (110) can identify a second hypergraph and input the second hypergraph to the second embedding encoder (1103). The processor (110) (e.g., the second embedding encoder (1103)) can identify second target embedding based on the second hypergraph (S400). For example, the processor (110) (e.g., the second embedding vector encoder (1103)) can identify disease embedding vectors based on the disease hypergraph.

The processor (110) (e.g., the second embedding encoder (1103)) can identify the second target embedding by applying a second attention mechanism that updates each of the nodes and hyperedges included in the second hypergraph.

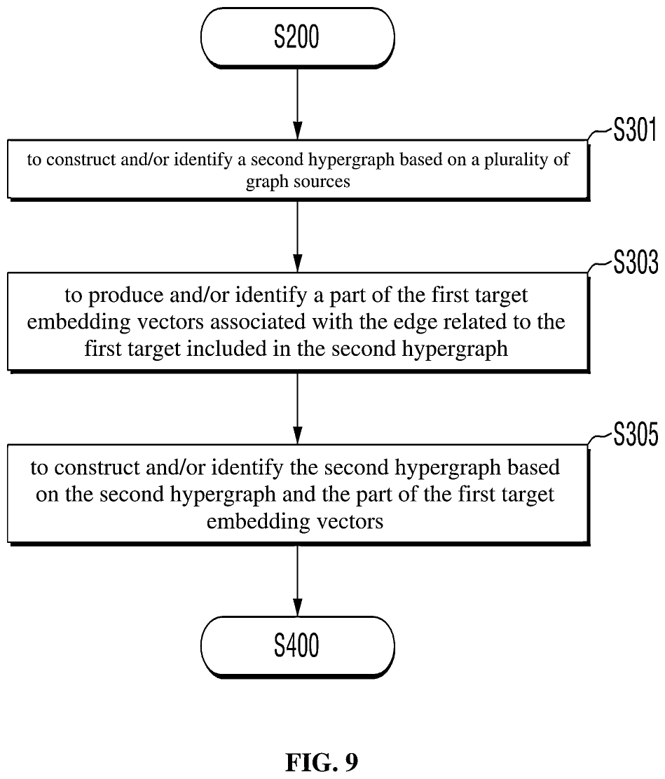

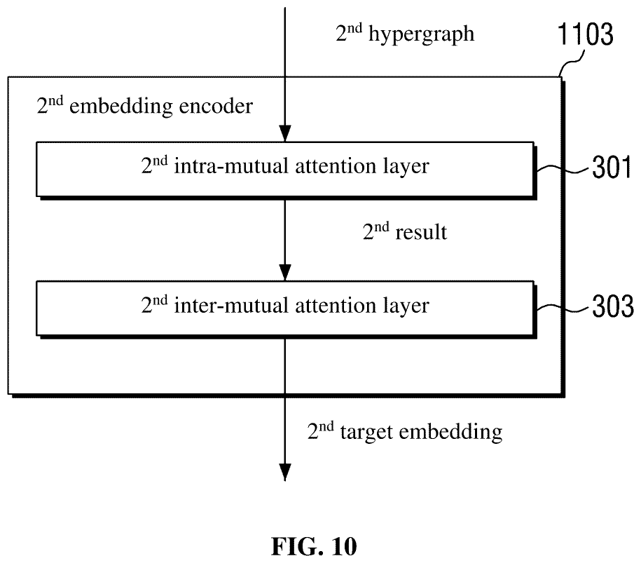

FIGS. 10-11 are drawings for explaining step S400 of FIG. 4.

Referring to FIGS. 1-5, 8, 10 and 11, a processor (110) (e.g., a second embedding encoder (1103)) can input a second hypergraph ((HG2) in FIG. 8) to a second intra mutual attention layer (301) to identify a second result.

The processor (110) (e.g., the second intra mutual attention layer (301) of the second embedding encoder (1103)) can identify a common relationship of at least one node included in a hyperedge of the second hypergraph (HG2) and update a hyperedge based on node information related to the common relationship (S401). In other words, the processor (110) (e.g., the second intra mutual attention layer (301) of the second embedding encoder (1103)) can integrate information of a node included in a hyperedge and update information of the hyperedge based on information of the integrated node.

For example, the processor (110) (e.g., the second intra mutual attention layer (301) of the second embedding encoder (1103)) can identify second node information related to a common relationship between four nodes included in the fourth hyperedge (E4) and update the fourth hyperedge (E4) based on the second node information. The four nodes can be different diseases. The processor (110) (e.g., the second intra mutual attention layer (301) of the second embedding encoder (1103)) can update at least one hyperedge each included in the second hypergraph (HG2) to output the second result.

In some embodiments, the second embedding encoder (1103) can include a third normalization layer and a third linear layer. For example, the second hypergraph (HG2) input to the second embedding encoder (1103) can be normalized through the third normalization layer, input to the second intra mutual attention layer (301), and output as a second result through the third linear layer.

The processor (110) (e.g., the second embedding encoder (1103)) can input the second result to the second inter mutual attention layer (303) to identify the second target embedding.

The processor (110) (e.g., the second inter mutual attention layer (303) of the second embedding encoder (1103)) can update a node of the second hypergraph (HG2) included in the second result. The processor (110) (e.g., the second intra mutual attention layer (301) of the second embedding encoder (1103)) can identify the second target embedding by updating at least one node included in the second hypergraph (HG2) based on the second node information (S403).

In some embodiments, the second embedding encoder (1103) can include a fourth normalization layer and a fourth linear layer. For example, the second result can be normalized through the fourth normalization layer, input to the second inter mutual attention layer (303), and output as second target embedding through the fourth linear layer. The second target embedding can include information about genes and diseases.

Referring again to FIGS. 1-4, the processor (110) (e.g., the integrated pair generation circuit (1104)) can identify at least one integrated pair based on the second target embedding (S500). The processor (110) (e.g., the integrated pair generation circuit (1104)) can identify an integrated pair between the first target (e.g., the gene) and the second target (e.g., the disease) based on the second target embedding generated including information about the first target (e.g., the gene) and the second target (e.g., the disease).

An integrated pair can include, for example, a first target and a second target that are presumed to be related to each other being associated with each other. For example, when the first target is a gene and the second target is a disease, if a specific disease is determined to be associated with a specific gene, the specific gene and the specific disease can be identified as a pair. For example, when the first target is a gene and the second target is a disease, if a specific gene is determined to function as a biomarker for a specific disease, the specific gene and the specific disease can be identified as a pair. An integrated pair can include, for example, a first target and a second target that are presumed to be very unrelated to each other being associated with each other. For example, when the first target is a gene and the second target is a disease, if a specific gene and a specific disease are determined to be unrelated to each other, the specific gene and the specific disease can be identified as a pair.

The processor (110) (e.g., the classification circuit (1105)) can classify at least one integrated pair based on at least one criterion (S600). For example, the processor (110) (e.g., the classification circuit (1105)) can classify at least one integrated pair based on a first criterion, a second criterion, and a third criterion. The first criterion can, for example, indicate that they are not related to each other or that not applicable (NA). The second criterion can, for example, indicate a biomarker. The third criterion can, for example, indicate a therapeutic gene.

A classification apparatus and method using a hypergraph according to an embodiment of the present invention generates disease embedding using a disease hypergraph including a part of gene embedding (one or more gene embedding vectors among the entire gene embedding vectors) which is a result of a first attention mechanism for a gene hypergraph, generates a gene-disease pair based on the disease embedding, and classifies the gene-disease pair according to a criterion. As a result, the accuracy of gene-disease classification can be improved, various characteristics of genes can be discovered, and possible treatment targets can be identified.

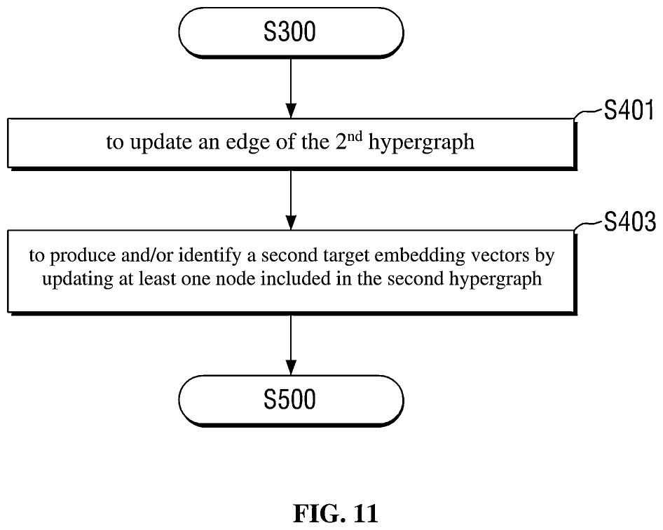

FIG. 12 is a flowchart for explaining a classification method using a hypergraph according to an embodiment of the present invention. For clarity of explanation, any part that overlaps with what has been explained previously is simplified or omitted.

A classification method using a hypergraph according to an embodiment of the present invention can include a step (S1000) of identifying a first hypergraph related to a first target. The first hypergraph can be identified based on a plurality of graph sources including graphs in which the first information and the second information each include a first node (e.g., gene node) and a second node (e.g., a gene ontology node), and the first node and the second node are connected by an edge. The first target can be, for example, a gene, and the second target can be, for example, a disease.

A classification method using a hypergraph according to an embodiment of the present invention can include a step (S2000) of identifying first target embedding based on a first hypergraph. In order to identify the first target embedding, a first attention mechanism can be applied to the first hypergraph. Based on first node information related to a common relationship of at least one first node included in a first hyperedge of the first hypergraph, the first hyperedge can be updated, and the at least one node included in the first hypergraph can be updated based on the updated first hyperedge information, so that first pre-target embedding can be identified. In other words, the first pre-target embedding (vectors) may refer to data produced by updating the hyperedge and the at least one hypernode included in the first hypergraph.

Based on that a first target type corresponds to a node and a hyperedge simultaneously among the first targets included in the first hypergraph, integrated embedding for the first target type can be identified, and the first target embedding can be identified based on the integrated embedding and the first pre-target embedding. For example, a gene A may be implemented as both a hypernode (vector) and a hyperedge (vector), and during the learning process of the AI model, two representations may be produced: a learned node vector and a learned hyperedge vector. As both vectors represent one gene A, the two vectors may be ‘integrated’ based on importance, and the resulting vectors may be defined as ‘integrated embedding (vectors).’

In some embodiments, a step can be performed of further including information related to the first hypergraph in the first target embedding.

A classification method using a hypergraph according to an embodiment of the present invention can include a step (S3000) of identifying a second hypergraph including a part of a first target embedding (one or more of the first target embedding vectors among the entire first target embedding vectors) and related to a second target. A second hypergraph can be identified based on a plurality of graph sources, a part (one or more) of a first target embedding related to a hyperedge (a hyperedge of the second target) related to the first target included in the second hypergraph can be identified, and the second hypergraph can be identified based on the second hypergraph and a part (one or more) of the first target embedding. As stated herein, the first target can be gene(s) and the second target can be disease(s).

A classification method using a hypergraph according to an embodiment of the present invention can include a step (S4000) of identifying a second target embedding based on a second hypergraph. Based on second node information related to a common relationship of at least one second node included in a second hyperedge of the second hypergraph, the second hyperedge is updated, and based on updated second hyperedge information, the at least one node included in the second hypergraph is updated, thereby produce and/or identifying the second target embedding.

A classification method using a hypergraph according to an embodiment of the present invention can include a step (S5000) of identifying at least one integrated pair based on second target embedding. For example, at least one gene-disease pair can be identified based on disease embedding that includes a part of gene embedding (one more gene embedding vectors among the entire gene embedding vectors).

A classification method using a hypergraph according to an embodiment of the present invention can include a step (S6000) of classifying at least one integrated pair based on at least one criterion. For example, a gene-disease pair can be classified based on a first criterion indicating that they are not related to each other, a second criterion indicating a biomarker, and a third criterion indicating a therapeutic gene.

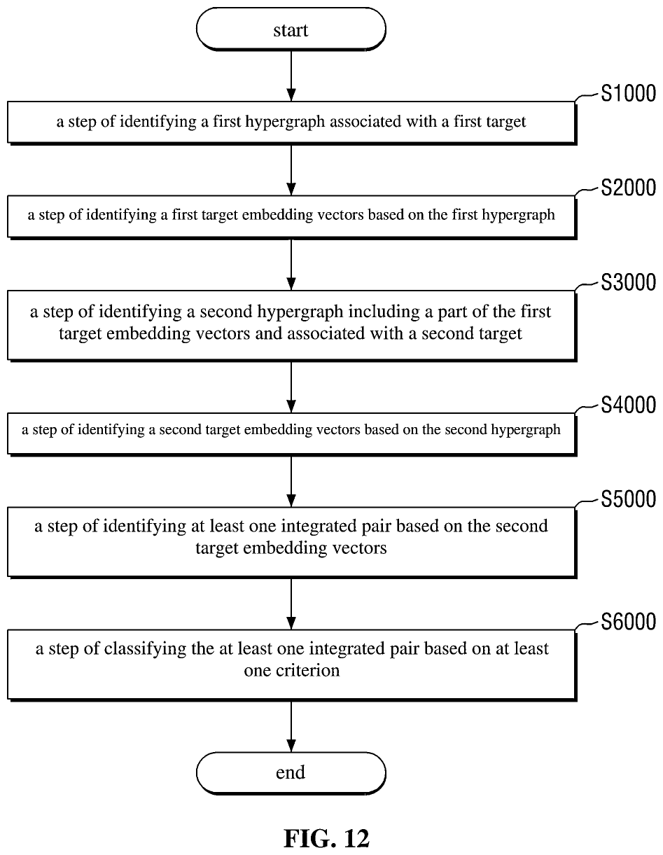

FIGS. 13 to 15 are drawings for explaining the effects of a classification apparatus and a method using a hypergraph according to an embodiment of the present invention.

FIG. 13 is a result table obtained for the classification performance evaluation index (F1) of each of comparative examples 1 to 5 and the Example with respect to accuracy, the first criterion (not-applicable or NA), the second criterion (biomarker), and the third criterion (therapeutic). In the case of comparative examples 1 to 5, each is a result of generating and classifying a gene-disease pair using a different classification model, or classifying the generated gene-disease pair. In the case of comparative examples 1 to 5, disease embedding is generated based on a hypergraph including at least a part of gene embedding (one or more gene embedding vectors among the entire gene embedding vectors), and gene-disease pairs are not generated based on the disease embedding vectors.

In the case of the embodiment, it can be seen that the classification performance evaluation index (F1) is close to 1 for accuracy, the first criterion (not applicable or NA), the second criterion (biomarker), and the third criterion (therapeutic) compared to the first to fifth comparative examples.

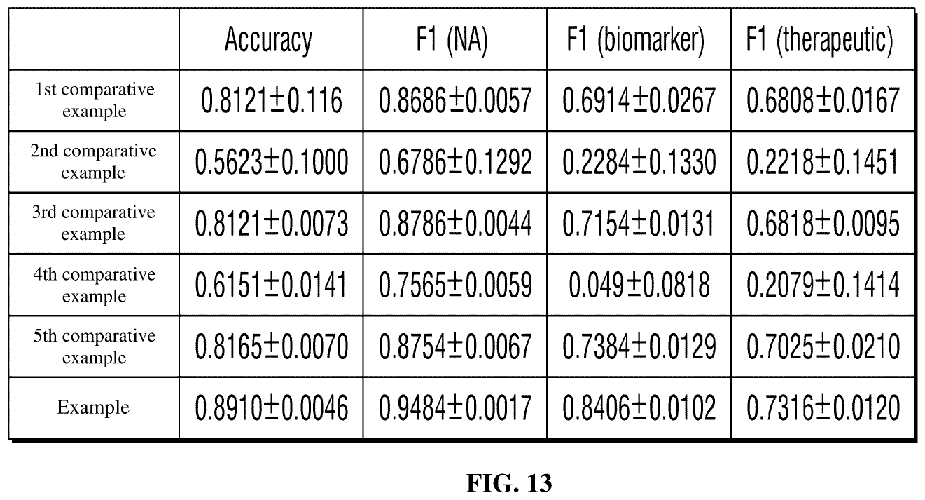

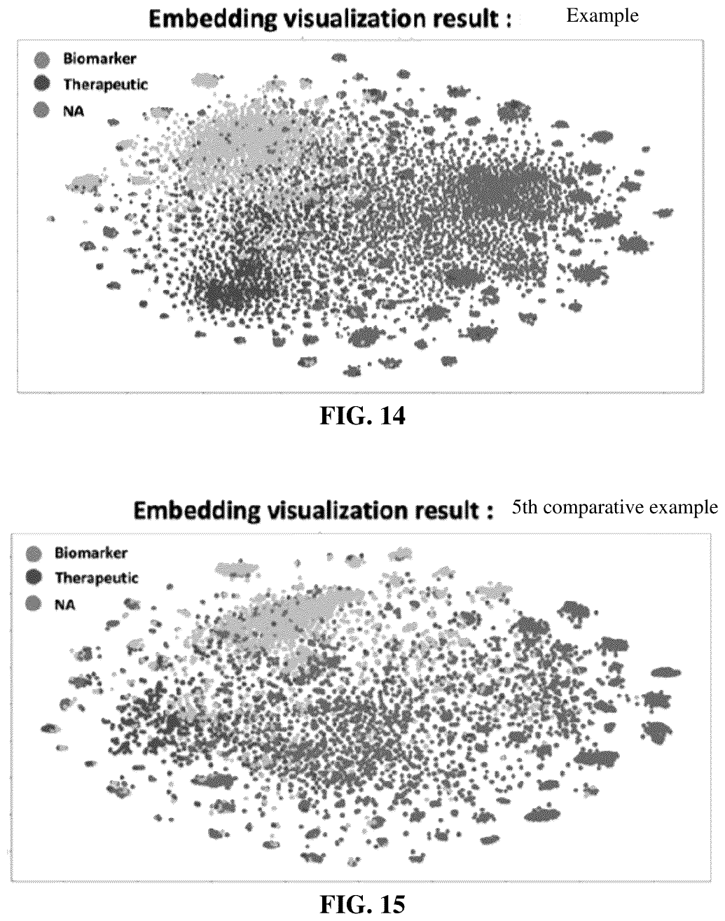

FIG. 14 is a result graph visually representing the embedding for an embodiment of the present invention. In FIG. 14, the blue portion indicates biomarker genes, the red portion indicates therapeutic target genes, and the gray portion indicates not applicable. FIG. 15 is a result graph visually representing the embedding for the fifth comparative example of FIG. 13. The fifth comparative example is a result of classifying gene-disease pairs by using a classification model that does not generate gene-disease pairs based on disease embedding by generating disease embedding based on a hypergraph that includes at least a part of gene embedding (one or more gene embedding vectors among the gene embedding vectors). In FIG. 15, the blue portion indicates biomarker genes, the red portion indicates therapeutic target genes, and the gray portion indicates not applicable.

Comparing FIGS. 14 and 15, in FIG. 14, the biomarkers and therapeutic targets form clear and distinct clusters in the embedding, which is the classification result of the gene-disease pair (e.g., integrated pair), whereas in FIG. 15, the biomarkers and therapeutic targets are dispersed in the embedding, which is the classification result of the gene-disease pair (e.g., integrated pair), and are also mixed with the first criterion indicating ‘unrelated’ or ‘not applicable’ (NA).

A classification apparatus and a method using a hypergraph according to an embodiment of the present invention can improve the accuracy of distinguishing between biomarker genes and therapeutic target genes by generating disease embedding using a disease hypergraph including a part of gene embedding (one or more embedding vectors among the entire gene embedding vectors) that is a result of a first attention mechanism for a gene hypergraph.

A classification apparatus and a method using a hypergraph according to an embodiment of the present invention generates disease embedding using a disease hypergraph including a part of gene embedding (one or more gene embedding vectors among the entire gene embedding vectors) which is a result of a first attention mechanism for a gene hypergraph, generates a gene-disease pair based on the disease embedding, and classifies the gene-disease pair according to a criterion, thereby obtaining an embedding result for a clearly distinguished gene-disease pair.

Various embodiments of the present document can be implemented as software (e.g., a program) including one or more instructions stored in a storage medium (e.g., a memory (120)) readable by a machine (e.g., a classification apparatus (100)). For example, a processor (e.g., a processor (110)) of the machine (e.g., a classification apparatus (100)) can load and execute at least one instruction among the one or more instructions stored to the storage medium. This enables the machine to operate to perform at least one function according to the at least one called instruction. The one or more instructions can include code generated by a compiler or code executable by an interpreter. The machine-readable storage medium can be provided in the form of a non-transitory storage medium. Here, ‘non-transitory’ simply means that the storage medium is a tangible device and does not contain signals (e.g. electromagnetic waves), and the term does not distinguish between cases where data is stored semi-permanently or temporarily on the storage medium.

According to one embodiment, the method according to the various embodiments disclosed in the present document can be provided as included in a computer program product. The computer program product may be traded between a seller and a buyer as a commodity. The computer program product may be distributed in the form of a machine-readable storage medium (e.g., a compact disc read only memory (CD-ROM)), or may be distributed online (e.g., downloaded or uploaded) via an application store (e.g., Play Store™) or directly between two user devices (e.g., smartphones). In the case of online distribution, at least a part of the computer program product may be at least temporarily stored or temporarily generated in a machine-readable storage medium, such as a memory of a manufacturer's server, a server of an application store, or an intermediary server.

According to various embodiments, each of the components (e.g., modules or programs) described above may include a single or multiple entities. According to various embodiments, one or more of the components or operations of the aforementioned components may be omitted, or one or more other components or operations may be added. Alternatively, or additionally, a plurality of components (e.g., modules or programs) may be integrated into a single component. In such a case, the integrated component may perform one or more functions of each of the components of the plurality of components identically or similarly to those performed by the corresponding component of the plurality of components prior to the integration. According to various embodiments, the operations performed by the modules, programs or other components may be executed sequentially, in parallel, repeatedly, or heuristically, or one or more of the operations may be executed in a different order, omitted, or one or more other operations may be added.

The above description is merely an example of the technical idea of the present embodiment, and those skilled in the art will appreciate that various modifications and variations may be made without departing from the essential characteristics of the present embodiment.

Accordingly, the present embodiments are not intended to limit the technical idea of the present embodiment, but rather to explain it, and the scope of the technical idea of the present embodiment is not limited by these embodiments. The protection scope of the present embodiment should be interpreted by the following claims, and all technical ideas within a scope equivalent thereto should be interpreted as being included in the scope of the rights of the present embodiment.

1. A therapeutic gene identification apparatus identifying through classification, the apparatus comprising:
at least one processor; and
at least one memory including a computer program code,
wherein the computer program code, when executed by the at least one processor, is configured, with the at least one processor, to cause the apparatus at least to:
construct a gene hypergraph associated with genes, the gene hypergraph comprising first hypernodes and first hyperedges;
produce gene embedding vectors by applying a first attention mechanism to the gene hypergraph, wherein the first attention mechanism is configured to update each of the first hypernodes and the first hyperedges;
construct a disease hypergraph including one or more of the gene embedding vectors, second hypernodes and second hyperedges, and associated with diseases;
produce disease embedding vectors which include information associated with the genes and the diseases by applying a second attention mechanism to the disease hypergraph, wherein the second attention mechanism is configured to update each of the second hypernodes and the second hyperedges; and
identify at least one gene-disease pair based on the disease embedding vectors,
wherein the computer program code is configured to cause the at least one processor to classify the at least one gene-disease pair based on a criterion including unrelated, a biomarker, and a therapeutic gene.

2. The therapeutic gene identification apparatus according to claim 1,
wherein the computer program code is configured to cause the at least one processor to:
construct the gene hypergraph based on a plurality of graph sources comprising nodes representing gene information and gene ontology information, wherein the nodes are connected by an edge in the plurality of graph sources.

3. The therapeutic gene identification apparatus according to claim 2,
wherein the computer program code is configured to cause the at least one processor to:
construct the disease hypergraph based on the plurality of graph sources;
produce one or more of the gene embedding vectors associated with at least one of the second hyperedges, wherein the at least one of the second hyperedges is related to the genes represented in the disease hypergraph; and
construct the disease hypergraph based on the disease hypergraph and the one or more of the gene embedding vectors.

4. The therapeutic gene identification apparatus according to claim 1,
wherein the computer program code is configured to cause the at least one processor to:
update the first hyperedges based on first node information associated with a common relationship of the first hypernodes included in the respective first hyperedges, and
produce pre-gene embedding vectors by updating the first hypernodes included in the gene hypergraph based on updated first hyperedge information.

5. The therapeutic gene identification apparatus according to claim 4,
wherein the computer program code is configured to cause the at least one processor to:
produce integrated embedding vectors for a gene among the genes, wherein information of the gene is included in both one of the first hypernodes and one of the first hyperedges; and
produce the gene embedding vectors based on the integrated embedding vectors and the pre-gene embedding vectors.

6. The therapeutic gene identification apparatus according to claim 5,
wherein the gene embedding vectors include information associated with the gene hypergraph.

7. The therapeutic gene identification apparatus according to claim 4,
wherein the computer program code is configured to cause the at least one processor to:
update the second hyperedges based on second node information associated with a common relationship of the second hypernodes included in the respective second hyperedges of the disease hypergraph; and
produce the disease embedding vectors by updating the second hypernodes based on updated second hyperedge information.

8. A therapeutic gene identification apparatus through classification, the apparatus comprising:
at least one processor; and
at least one memory including a computer program code,
wherein the computer program code, when executed by the at least one processor, is configured, with the at least one processor, to cause the apparatus at least to:
construct a gene hypergraph associated with genes, the gene hypergraph comprising first hypernodes and first hyperedges;
update the first hyperedges included in the gene hypergraph based on first node information associated with a common relationship of the first hypernodes included in the respective first hyperedges;
produce pre-gene embedding vectors by updating the first hypernodes included in the gene hypergraph based on updated first hyperedge information;
produce integrated embedding vectors for a gene among the genes, wherein information of the gene is included in both one of the first hypernodes and one of the first hyperedges;
produce gene embedding vectors based on the integrated embedding vectors and the pre-gene embedding vectors;
construct a disease hypergraph including one or more of the gene embedding vectors, second hypernodes, and second hyperedges, and associated with diseases;
update the second hyperedges based on second node information associated with a common relationship of the second hypernodes included in the respective second hyperedges of the disease hypergraph;
produce disease embedding vectors including information associated with the genes and the diseases by updating the second hypernodes based on updated second hyperedge information;
identify at least one gene-disease pair based on the disease embedding; and
classify the at least one gene-disease pair based on at least one criterion including unrelated, a biomarker, and a therapeutic gene.

9. The therapeutic gene identification apparatus according to claim 8,
wherein the computer program code is configured to cause the at least one processor to:
construct the gene hypergraph based on a plurality of graph sources comprising nodes representing gene information and gene ontology information, wherein the nodes are connected by an edge in the plurality of the graph sources.

10. The therapeutic gene identification apparatus according to claim 9,
wherein the computer program code is configured to cause the at least one processor to:
construct the disease hypergraph based on the plurality of graph sources;
produce one or more of the gene embedding vectors associated with at least one of the second hyperedges, wherein the at least one of the second hyperedges is related to the genes represented in the disease hypergraph; and
construct the disease hypergraph based on the disease hypergraph and the one or more of the gene embedding vectors.

11. The therapeutic gene identification apparatus according to claim 8,
wherein the gene embedding vectors include information associated with the gene hypergraph.

12. A therapeutic gene identification method, the method comprising:
identifying a gene hypergraph associated with genes, the gene hypergraph comprising first hypernodes and first hyperedges;
identifying gene embedding vectors by applying a first attention mechanism to the gene hypergraph, wherein the first attention mechanism is configured to update each of the first hypernodes and the first hyperedges;
identifying a disease hypergraph including one or more of the gene embedding vectors, second hypernodes, and second hyperedges, and associated with diseases;
identifying disease embedding vectors including information associated with the genes and the diseases by applying a second attention mechanism to the disease hypergraph, wherein the second attention mechanism is configured to update each of the second hypernodes and the second hyperedges;
identifying at least one gene-disease pair based on the disease embedding vectors; and
classifying the at least one gene-disease pair based on a criterion including unrelated, a biomarker, and a therapeutic gene.

13. The therapeutic gene identification method according to claim 12,
wherein the identifying of the gene hypergraph is performed based on a plurality of graph sources comprising nodes representing gene information and gene ontology information, wherein the nodes are connected by an edge in the plurality of graph sources.

14. The therapeutic gene identification method according to claim 13,
wherein the identifying of the disease hypergraph is performed based on the plurality of graph sources, and the identifying of the disease hypergraph includes:
identifying one or more of the gene embedding vectors associated with at least one of the second hyperedges, wherein the at least one of the second hyperedges is related to the genes represented in the disease hypergraph; and
identifying the disease hypergraph based on the disease hypergraph and the one or more of the gene embedding vectors.

15. The therapeutic gene identification method according to claim 12,
wherein the identifying of the gene embedding vectors further includes applying the first attention mechanism so as to:
update the first hyperedges based on first node information associated with a common relationship of the first hypernodes included in the respective first hyperedges; and
identify pre-gene embedding vectors by updating the first hypernodes included in the gene hypergraph based on updated first edge information.

16. The therapeutic gene identification method according to claim 15,
wherein the identifying of the gene embedding vectors includes:
identifying integrated embedding vectors for a gene among the genes, wherein information of the gene is included in both one of the first hypernodes and one of the first hyperedges; and
identifying the gene embedding vectors based on the integrated embedding vectors and the pre-gene embedding vectors.

17. The therapeutic gene identification method according to claim 16,
the method further comprising: including information associated with the gene hypergraph to the gene embedding vectors.

18. The therapeutic gene identification method according to claim 15,
wherein the identifying of the disease embedding vectors includes applying the second attention mechanism so as to:
update the second hyperedges based on second information associated with a common relationship of the second hypernodes included in the respective second hyperedges of the disease hypergraph; and
identify the disease embedding vectors by updating the second hypernodes based on updated second edge information.
